(19) United States
(12) Patent Application Publication
(10) Pub. No.: US 2023/0330163 A1
(43) Pub. Date: Oct. 19, 2023

(54) METHOD FOR REDUCING BEHAVIORAL ABNORMALITIES

(71) Applicant: BENED BIOMEDICAL CO., LTD., Taipei City (TW)

(72) Inventors: Ying-Chieh TSAI, Taipei city (TW); Chin-Lin HUANG, Taipei City (TW); Chien-Chen WU, Taipei City (TW); Chih-Chieh HSU, Taipei City (TW)

(73) Assignee: BENED BIOMEDICAL CO., LTD., Taipei City (TW)

(21) Appl. No.: 17/052,227

(22) PCT Filed: Mar. 25, 2020
(86) PCT No.: PCT/CN2020/081020

§ 371 Date: Nov. 2, 2020

(51) Int. Cl. A61K 35/747; A61K 31/519; A61K 31/496; A61K 31/138; A61P 25/00

(52) U.S. Cl. CPC A61K 35/747; A61K 31/519; A61K 31/496; A61K 31/138; A61P 25/00

(57) ABSTRACT

Provided is a method of reducing a behavioral abnormality associated with a neurodevelopmental disorder in a subject, including administering to the subject an effective amount of Lactobacillus plantarum subsp. plantarum PS128. Also provided is a composition for preventing or treating a behavioral abnormality associated with a neurodevelopmental disorder in a subject in need thereof.

### BACKGROUND

### 1. Technical Field

The present disclosure relates to a method of reducing behavioral abnormalities in a subject. More particularly, it relates to the use of Lactobacillus plantarum subsp. plantarum PS128 to improve the behavior of an individual suffering from a condition or a disease associated with a neurodevelopmental disorder.

### 2. Description of Associated Art

Individuals with neurodevelopmental disorders are characterized by a combination of abnormal behaviors and mental conditions, such as restricted, repetitive or stereotypic behaviors, interests or activities, attention deficits, oppositional and defiant behaviors, and deficits in communication and social interactions. These neurodevelopmental disorders include, for example, autism spectrum disorder (ASD), attention deficit hyperactivity disorder (ADHD), oppositional defiant disorder (ODD), and Tourette syndrome (TS).

For the treatment of such neurodevelopmental disorders, antipsychotics, e.g., risperidone and aripiprazole, were approved by the U.S. FDA to treat ADHD symptoms and irritability associated with autistic disorders for patients aged from 5 to 16 and from 6 to 17 years old, respectively. However, these drugs fail to treat other abnormal behaviors effectively, and, therefore, FDA has not yet approved the drugs for the indications associated with the core symptoms of ASD such as persistent deficits in communication and social interactions accompanied by restricted and repetitive patterns of behaviors, interests, and activities. While not all individuals respond to the drugs, there are several associated sides effects, such as increase in appetite, drowsiness and hormonal changes, which make the drugs undesirable and inadequate.

In addition to the use of antipsychotics, the mainstream interventions for alleviating ASD symptoms involve psychosocial strategies, such as educational interventions, speech therapies, behavioral interventions, developmental therapies and parenting skill training programs, with varying effects on improving communication and social behaviors. Dietary and nutritional supplements, such as ketogenic diet, vitamins, fatty acids, and gluten-free, were reportedly to have certain effects on various ASD symptoms. However, no sufficient evidence found was adequate to support the efficacy on ASD.

Therefore, there remains a need for a safe and effective method to reduce and ameliorate behavioral abnormalities associated with neurodevelopmental disorders. Such reduction of behavioral abnormalities in individuals with neurodevelopmental disorders is important and greatly beneficial in that therapies; trainings and education could be conducted more effectively with less disturbances, and therefore are better assimilated and adopted by the individuals, consequently improving the quality of their lives.

### SUMMARY

In view of the foregoing, the present disclosure provides a method for reducing one or more behavioral abnormalities in a subject suffering from a neurodevelopmental disorder, comprising administering a composition comprising an effective amount of Lactobacillus plantarum subsp. plantarum PS128. In one embodiment, the effective amount of PS128 is at least 1×10⁹ CFU, at least 1×10¹⁰ CFU or at least 1×10¹¹ CFU, including 2×10⁹ CFU, 3×10⁹ CFU, 4×10⁹ CFU, 5×10⁹ CFU, 6×10⁹ CFU, 7×10⁹ CFU, 8×10⁹ CFU, 9×10⁹ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰, 2×10¹¹ CFU, 3×10¹¹ CFU, 4×10¹¹ CFU, 5×10¹¹ CFU, 6×10¹¹ CFU, 7×10¹¹ CFU, 8×10¹¹ CFU, and 9×10¹¹ CFU, but not limited thereto.

In one embodiment of the present disclosure, the subject is an individual suffering from a neurodevelopmental disorder and aged between 7 and 15 years old. In another embodiment of the present disclosure, the individual suffering from a neurodevelopmental disorder is aged between 7 and 12 years old. In another embodiment, the individual suffering from a neurodevelopmental disorder is aged 7, 8, 9, 10, 11, 12, 13, 14 or 15 years old. In yet another embodiment, the individual suffering from a neurodevelopmental disorder is aged 7, 8, 9, 10, 11 and 12 years old.

In one embodiment of the present disclosure, the composition is administered to the subject for at least 1 week, 2 weeks, 3 weeks, 4 weeks, 5 weeks or 6 weeks. In another embodiment, the composition is administered to the subject for at least 1 month, 2 months or 3 months.

In one embodiment of the present disclosure, the neurodevelopmental disorder is anxiety, autism, autism spectrum disorder (ASD), a mental condition with some of the symptoms of ASD, Tourette syndrome, obsessive compulsive disorder, attention deficit hyperactivity disorder, oppositional defiant disorder, Asperger's syndrome, childhood disintegrative disorder or Rett syndrome. In a further embodiment of the present disclosure, the neurodevelopmental disorder is autism or autism spectrum disorder (ASD).

In one embodiment of the present disclosure, the one or more behavioral abnormalities comprise withdrawal behavior, stereotyped behavior, repetitive behavior, compulsive behavior, aggressive behavior, rule-breaking behavior, deficit in social interaction, deficit in communication, deficit in attention, deficit in adaptability and self-care, deficit in social awareness, deficit in social emotion, abnormal sensation and perception, abnormal behavior in making relation and connection, abnormal body and object use behavior, hyperactive or impulsive behavior, oppositional or defiance behavior, or anxiety behavior.

In one embodiment of the present disclosure, the one or more behavioral abnormalities is evaluated by standard behavioral evaluation. In a further embodiment of the present disclosure, the standard behavioral evaluation comprises Aberrant Behavior Checklist-Taiwan version (ABC-T), Child Behavior Checklist (CBCL), Clinical Global Impression-Improvement (CGI-I), Clinical Global Impression-Severity (CGI-S), Swanson, Nolan and Pelham (SNAP)-IV-Taiwan version (SNAP-IV), Social Responsiveness Scale (SRS), or any combination thereof.

In one embodiment of the present disclosure, reducing one or more behavioral abnormalities comprises reducing anxiety level, reducing abnormal body and object use behavior, reducing rule-breaking behavior, reducing hyperactive or impulsive behavior, reducing oppositional or defiance behavior, reducing attention deficit, or any combination thereof. In another embodiment of the present disclosure, reducing one or more behavioral abnormalities comprises reducing oppositional or defiance behavior, reducing anxiety level, reducing rule-breaking behavior, reducing attention deficit, reducing hyperactive or impulsive behavior, or any combination thereof. In a further embodiment of the present disclosure, reducing one or more behavioral abnormalities results in improvement in mental age. In another embodiment, reducing one or more behavioral abnormalities comprises reducing dystonia.

In an aspect of the present disclosure, a method for treating a neurodevelopmental disorder in a subject in need thereof is provided, comprising administering a composition comprising an effective amount of Lactobacillus plantarum subsp. plantarum PS128 as a sole active ingredient for reducing one or more behavioral abnormalities associated with a neurodevelopmental disorder. In one embodiment of the present disclosure, the composition is orally administrated to the subject. In another embodiment, the method further comprising administering an antipsychotic in combination with the composition to the subject. In a further embodiment, the antipsychotic is selected from the group consisting of risperidone, aripiprazole, and atomoxetine hydrochloride. In yet another embodiment, the antipsychotic and the composition are administered to the subject at different time intervals.

In one embodiment of the present disclosure, the method further comprising administering an additional therapy to the subject. In a further embodiment, the additional therapy is selected from the group consisting of applied behavior analysis (ABA), developmental, individual differences, relationship-based approach (DIR), treatment and education of autistic and related communication-handicapped children (TEACCH), picture exchange communication system (PECS), sensory integration therapy, Floortime approach and a combination thereof.

In one embodiment of the present disclosure, the composition is a pharmaceutical composition further comprising a pharmaceutically acceptable carrier. In another embodiment of the present disclosure, the pharmaceutically acceptable carrier can be a physiologically acceptable excipient or diluent. In yet another embodiment of the present disclosure, the examples of the physiologically acceptable excipient or diluent include, but are not limited to, lactose, starch, dextrin, cyclodextrin, sodium carboxymethyl starch, carboxylated starch propionate, microcrystalline cellulose, carboxymethyl cellulose, maltodextrin and magnesium stearate.

### DETAILED DESCRIPTION OF THE EMBODIMENTS

The following embodiments are used to exemplify the present disclosure. A person of ordinary skill in the art can conceive the other advantages of the present disclosure, based on the specification of the present disclosure. The present disclosure can also be implemented or applied as described in different embodiments. It is possible to modify and/or alter the examples for carrying out this disclosure without contravening their spirit and scope, for different aspects and applications.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed descriptions of the present disclosure. Thus, the terms used herein have to be defined based on the meaning of the terms together with the descriptions throughout the specification.

Also, when a part “includes” or “comprises” a component or a step, unless there is a particular description contrary thereto, the part can further include other components or other steps, not excluding the others.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

The phrase “an effective amount” refers to the amount of an active ingredient that is required to result in a reduction, inhibition or prevention of the behavioral disorder, abnormality or symptom in the individual. An effective amount will vary, as recognized by those skilled in the art, depending on routes of administration, excipient usage, and the possibility of co-usage with other therapeutic treatment.

The term “individual” as used herein includes a single biological organism, of which a neurodevelopmental disorder may occur including, but not limited to, animals and in particular vertebrates such as mammals and in particular human beings.

The term “condition,” “disorder,” “symptom” or “behavioral abnormality” as used herein refers to symptoms expressed by an individual with a mental disorder, such as but not limited to anxiety, autism, autism spectrum disorders, Rett syndrome, Tourette syndrome, obsessive compulsive disorder, attention deficit hyperactivity disorder, oppositional defiant disorder, Asperger's syndrome, or childhood disintegrative disorder.

The term “individual in need of the treatment” refers to a person expressing or suffering from one or more of the behavioral disorders or symptoms mentioned above. An appropriately qualified person is able to identify such an individual in need of treatment using standard behavioral testing protocols or guidelines. The same behavioral testing protocols or guidelines may also be used to determine whether there is improvement to the individual's disorders or symptoms, or determine the most effective dose of PS128 to be administered to an individual in need of the treatment.

The term “improvement in behavioral performance” as used herein refers to prevention or reduction in the severity or frequency, to whatever extent, of one or more of the above behavioral disorders, symptoms or abnormalities expressed by the individual. The improvement is either observed by the individual taking the treatment themselves or by another person.

Different examples have been used to illustrate the present disclosure. The examples below should not be taken as a limit to the scope of the present disclosure.

### EXAMPLE

The present disclosure examined the effects of Lactobacillus plantarum subsp. plantarum PS128 for the treatment of neurodevelopmental disorders.

A randomized, double-blind, placebo-controlled trial was conducted in Taiwan with the recruitment of 80 subjects. Eligible subjects were randomly allocated into the two arms of the study in a 1:1 ratio, according to randomly permuted blocks within the strata of two assignments, the probiotic group (PS128) and placebo group, using treatment codes. Randomization was performed by a research assistant who had no contact with the participants. The allocation sequence was not available to any member of the research team, the physician or the participants. The primary outcomes of this study were changes in the Autism Behavior Checklist-Taiwan version (ABC-T) questionnaire, the Social Responsiveness Scale (SRS) scores, and the Child Behavior Checklist (CBCL) questionnaire, and the secondary outcomes were improvement in the Chinese version of the Swanson, Nolan, and Pelham-IV (SNAP-IV) assessment and the Clinical Global Impression-Improvement (CGI-I). During participation in this trial, all subjects were further confirmed with the diagnosis of ASD according to the Autism Diagnostic Interview-Revised (ADI-R) performed by trained testers.

### Participants

Participants in the study were screened based on inclusion and exclusion criteria. The inclusion criteria were boys aged from 7 to 15 years old diagnosed with ASD based on the Diagnostic and Statistical Manual of Mental Disorders, 5th edition (DSM-V) criteria. Major caregiver was asked to provide the physical and mental disability card provided by the Taiwan government, and the researcher of this study checked the ICD-9 (International Classification of Diseases, Ninth Revision) code to be 299.00. Considering the prevalence of ASD is about four times more common among boys than among girls, only boys with ASD were included into this study. The exclusion criteria included the consumption of prescribed antibiotics and yogurt or probiotic products two weeks prior to enrollment. Participants were allowed to continue their regular medications, treatment and therapies, with the exception of antibiotics, and were asked to refrain from consuming yogurt or probiotic products during the study period. Written informed consent was obtained from all subjects and the parents or caregivers of subjects prior to the start of the study.

### PS128 and Placebo Products

Lactobacillus plantarum PS128 used in the study was the strain isolated and deposited under DSMZ Accession No. DSM 28632. The PS128 provided to the participants was in a form of capsules containing creamy white powder. The probiotic capsules weighed 425+25 mg and contained 3×10¹⁰ CFU/capsule of PS128 with microcrystalline cellulose as the carrier, whereas the placebo capsules only contained microcrystalline cellulose. All capsules were identical in taste and appearance and were stored at a refrigerated temperature (4 to 8° C.).

### Assessments

The Clinical Global Impression-Severity (CGI-S) and Clinical Global Impression-Improvement (CGI-I) forms were completed by medical professionals at the baseline (week 0) and upon completion of the intervention (week 4), respectively. Parents or caregiver completed subsequent scores for the ABC-T, CBCL, SRS, and SNAP-IV questionnaires. This study comprised two visits, where the baseline visit involved enrollment, randomization prior to the start of the study, and assessments using questionnaires, whereas the second visit involved a review of the subject's medical history and reports of adverse events (week 4).

The ABC-T is a 47-item questionnaire used to assess behavioral problems in children with intellectual and developmental disabilities which was modified from Autism Behavior Checklist of Autism Screening Instrument for Education Planning-Third edition. This validated tool is divided into five subscales, including problems related to sensory (sensation and perception; eight items), relating (relation and connection; 11 items), body and object use (physical activity and rigid use of objects; 12 items), language (communication and interaction; eight items) and social and self-help (adaptability and self-care; eight items). ABC-T items were rated as “yes” (rated as 1, with symptom) or “no” (rated as 0, without symptom) for each question during the assessment.

SRS is a validated 65-item assessment tool designing to assess social communication and interactions, as well as restricted interests and repetitive behaviors. These assessments are categorized into social awareness, social communication, social emotion and autistic mannerisms.

CBCL is a 113-item questionnaire which assesses eight empirically based syndrome scales, including aggressive behaviors, anxiousness, attention problems, rule-breaking behavior, somatic complaints, social problems, thought problems and withdrawal issues.

CGI-S and CGI-I questionnaires both comprise items measured on a seven-point scale that are rated by a clinician to determine symptom severity and improvement, respectively.

SNAP-IV was used to evaluate the ADHD and ODD in children aged from 6 to 15 years old. It is useful for interpretation of the effects of interventions on patients enrolled in clinical studies. The Taiwan version of the SNAP-IV consisted of 26 items reflecting DSM-IV symptoms of attention-deficit hyperactivity disorder (ADHD) (18 items) and oppositional and defiance problems (8 items).

### Data Analysis

Data were analyzed using GraphPad Prism (Version 7; GraphPad Software, San Diego, CA, USA) and Excel.

Two-tailed t-test was conducted to compare demographics (parametric test), clinical characteristics (ADI-R scores, CGI-S, and CGI-I) and scores of outcome measurements between the PS128 group and placebo group at baseline and week 4.

For the exploratory analysis stratified by age, the subjects were divided into the age groups of from 7 to 12 and from 13 to 15 years old. Independent t-test was applied to compare the differences of questionnaires (the week 4 score and baseline score) between the PS128 and placebo groups. Scores of baseline and week 4 were analyzed by using a paired t-test for within-group analysis. A two-tailed significance level of P <0.05 was considered statistically significant.

### Results

A total of 80 subjects were recruited and randomly assigned to treatment: 39 assigned to PS128 and 41 assigned to placebo. Subjects were confirmed for the diagnosis of ASD by ADI-R assessment by experienced researchers. Among the eligible 80 subjects aged between 7 and 15 years old, 3 and 6 subjects dropped out within the PS128 and placebo groups, respectively, yielding 71 subjects at the end of the study period (PS128, n=36; placebo, n=35). No adverse event was reported in this study. No subjects or their parents reported any gastrointestinal intolerance or allergic responses during their participation.

As shown in Table 1 below, the demographic data including age, height and weight and clinical characteristics were similar among the PS128 and placebo group (p>0.05). Subjects from both groups fulfilled the criteria for autism, with scores exceeding the cut-off values for all four domains, i.e., reciprocal social interaction, language and communication (verbal and non-verbal), stereotyped repetitive behaviors or interests, and age-of-onset, and both scored similarly (P>0.05). The CGI-S scores of the PS128 and placebo groups were also similar at baseline (P=0.26).

TABLE 1
Demographic and clinical characteristics of the subjects at baseline

| Characteristic | PS128 | Placebo | Total | P-Value |
|---|---|---|---|---|
| Sample size (n) | 36 | 35 | 71 | |
| Age | 10.11 | 9.91 | 10.01 | 0.72 |
| | (2.34) | (2.33) | (2.32) | |
| Height | 144.16 | 140.63 | 142.39 | 0.38 |
| | (16.31) | (15.44) | (15.85) | |
| Weight | 37.31 | 35.65 | 36.49 | 0.65 |
| | (13.56) | (15.12) | (14.26) | |
| CGI-S | 4.86 | 5.17 | 5.01 | 0.26 |
| | (1.25) | (1.04) | (1.15) | |

Autism Diagnostic Interview-Revised (ADI-R) Scores

| | | | | |
|---|---|---|---|---|
| Qualitative abnormalities in | 22.81 | 24.03 | 23.41 | 0.37 |
| reciprocal social interaction | (4.78) | (6.55) | (5.71) | |
| Qualitative abnormalities in | 16.22 | 16.20 | 16.21 | 0.98 |
| communication (Verbal) | (4.66) | (4.90) | (4.75) | |
| Qualitative abnormalities in | 9.19 | 9.20 | 9.20 | 0.99 |
| communication (Non-verbal) | (3.18) | (2.92) | (3.03) | |
| Restricted, repetitive, and | 7.11 | 8.14 | 7.62 | 0.08 |
| stereotyped patterns of behavior | (2.41) | (2.48) | (2.48) | |
| Abnormality of development | 3.86 | 3.83 | 3.85 | 0.92 |
| evident at or before 36 months | (1.38) | (1.49) | (1.42) | |

The results are expressed as means (SD).

The assessment results of CGI-I, ABC-T, SRS, CBCL and SNAP-IV were shown in Table 2 below. It showed that the CGI-I scores for both groups were equivalent to “minimally improved” (3.64 and 3.66 for the PS128 and placebo groups, respectively; P=0.94). There was no difference between the PS128 and placebo groups in the total and subscale scores of ABC-T, SRS, CBCL and SNAP-IV at both baseline and week 4.

TABLE 2
Assessment results of CGI-I, ABC-T, SRS, CBCL
and SNAP-IV over the 4-week treatment of PS128

| | Baseline | | | Week 4 | | |
|---|---|---|---|---|---|---|
| | PS128 | Placebo | P-Value | PS128 | Placebo | P-Value |
| CGI-I | | | | 3.64 | 3.66 | 0.94 |
| | | | | (1.1) | (1.00) | |

ABC-T

| | | | | | | |
|---|---|---|---|---|---|---|
| Sensory | 2.24 | 3.03 | 0.07 | 2.21 | 2.75 | 0.19 |
| | (1.41) | (1.98) | | (1.58) | (1.74) | |
| Relating | 3.88 | 3.74 | 0.82 | 4.03 | 3.63 | 0.59 |
| | (2.77) | (2.43) | | (3.08) | (2.74) | |
| Body and object use | 3.42 | 3.71 | 0.66 | 3.12 | 3.45 | 0.62 |
| | (2.62) | (2.71) | | (2.59) | (2.68) | |
| Language | 2.47 | 3.0 | 0.27 | 2.15 | 2.81 | 0.16 |
| | (2.15) | (1.77) | | (1.89) | (1.84) | |
| Social and self help | 3.24 | 3.59 | 0.48 | 3.15 | 3.52 | 0.52 |
| | (2.02) | (2.11) | | (1.98) | (2.45) | |
| Total score | 15.81 | 17 | 0.59 | 14.67 | 16.21 | 0.53 |
| | (8.39) | (9.31) | | (8.97) | (10.11) | |

SRS

| | | | | | | |
|---|---|---|---|---|---|---|
| Social communication | 64.06 | 64.0 | 0.99 | 62.17 | 63.34 | 0.75 |
| | (14.84) | (15.87) | | (12.9) | (16.21) | |
| Autism mannerisms | 30.82 | 30.39 | 0.79 | 29.97 | 29.71 | 0.88 |
| | (6.26) | (6.58) | | (7.25) | (6.53) | |
| Social awareness | 23.29 | 21.11 | 0.06 | 23.21 | 21.57 | 0.21 |
| | (4.22) | (5.27) | | (4.89) | (5.25) | |
| Social emotion | 19.74 | 19.11 | 0.56 | 19.24 | 18.81 | 0.72 |
| | (4.54) | (4.34) | | (4.55) | (5.14) | |
| Total score | 138.87 | 135.88 | 0.64 | 132.77 | 135.79 | 0.63 |
| | (24.19) | (26.04) | | (22.99) | (25.79) | |

CBCL

| | | | | | | |
|---|---|---|---|---|---|---|
| Anxiety | 6.76 | 6.0 | 0.51 | 5.63 | 5.84 | 0.84 |
| | (4.85) | (4.46) | | (4.34) | (4.41) | |
| Withdrawn | 4.41 | 4.68 | 0.71 | 4.22 | 4.50 | 0.7 |
| | (2.83) | (3.05) | | (2.67) | (3.16) | |
| Somatic complaints | 2.38 | 3.13 | 0.22 | 2.5 | 2.41 | 0.9 |
| | (1.79) | (2.92) | | (2.53) | (2.72) | |
| Internalization | 13.32 | 13.60 | 0.9 | 12.34 | 12.72 | 0.85 |
| | (7.98) | (8.46) | | (6.83) | (8.53) | |
| Social problems | 7.65 | 7.71 | 0.95 | 7.38 | 7.65 | 0.78 |
| | (3.98) | (3.13) | | (4.04) | (3.67) | |
| Thoughts problems | 5.73 | 6.90 | 0.35 | 4.84 | 6.50 | 0.17 |
| | (4.04) | (5.57) | | (3.96) | (5.28) | |
| Attention problems | 10.61 | 11.12 | 0.59 | 10.87 | 10.90 | 0.98 |
| | (3.74) | (3.92) | | (4.42) | (4.28) | |
| Rule-breaking behavior | 3.5 | 3.84 | 0.65 | 3.06 | 3.31 | 0.77 |
| | (3.3) | (2.74) | | (3.62) | (3.15) | |
| Aggressive behavior | 8.27 | 8.71 | 0.8 | 8.06 | 7.94 | 0.94 |
| | (7.0) | (6.21) | | (7.02) | (6.02) | |
| External | 11.73 | 12.75 | 0.67 | 11.1 | 11.25 | 0.95 |
| | (9.73) | (8.64) | | (10.2) | (8.47) | |
| Total score | 49.63 | 50.60 | 0.89 | 44.34 | 49.20 | 0.53 |
| | (25.4) | (25.91) | | (23.25) | (24.46) | |

SNAP-IV

| | | | | | | |
|---|---|---|---|---|---|---|
| Inattention | 15.18 | 15.79 | 0.66 | 14.39 | 15.35 | 0.5 |
| | (5.83) | (5.16) | | (5.91) | (5.48) | |
| Hyperactivity/impulsivity | 10.3 | 10.97 | 0.64 | 9.65 | 10.25 | 0.68 |
| | (5.51) | (6.0) | | (5.23) | (6.42) | |
| Opposition/defiance | 8.93 | 7.5 | 0.32 | 7.73 | 7.41 | 0.8 |
| | (6.09) | (5.41) | | (5.04) | (5.43) | |
| Total score | 34.03 | 34.48 | 0.9 | 31.87 | 33.16 | 0.73 |
| | (14.61) | (13.39) | | (14.26) | (15.58) | |

The results are expressed as means (SD).
ABC-T: Aberrant Behavior Checklist-Taiwan version;
CBCL: Child Behavior Checklist;
CGI-I: Clinical Global Impression-Improvement;
SNAP-IV: Swanson, Nolan and Pelham (SNAP)-IV-Taiwan version;
SRS: Social Responsiveness Scale.

However, when the assessment results were further stratified by age analysis (7 to 12 years old for elementary school; 13 to 15 years old for junior high school), there were obvious improvements in the PS128 group, as shown in Table 3 below.

In the elementary school subjects aged from 7 to 12 years old, the scores of social awareness of the placebo group were lower than the PS128 group at baseline (P=0.02) and also at week 4 (P=0.04). By comparing the changes over time (the week 4 score and baseline score), the PS128 group showed improved opposition/defiance (P=0.03) and total score (P=0.02) of SNAP-IV when compared with the placebo group.

TABLE 3
Age stratified analysis of outcomes for subjects aged from 7 to 12 years old

| | | | | | | | Difference |
|---|---|---|---|---|---|---|---|
| | | | | | | | between |
| | | | | | | | PS128 |
| | | | | | | | and |
| | Baseline | | | Week 4 | | | placebo |
| | PS128 | Placebo | P-Value | PS128 | Placebo | P-Value | P-Value |
| CGI-I | | | | 3.68 | 3.57 | 0.69 | |
| | | | | (1.14) | (1.04) | | |

ABC-T

| | | | | | | | |
|---|---|---|---|---|---|---|---|
| Sensory | 2.36 | 2.9 | 0.22 | 2.29 | 2.68 | 0.38 | 0.41 |
| | (1.47) | (1.88) | | (1.61) | (1.68) | | |
| Relating | 4.14 | 3.66 | 0.49 | 4.43 | 3.77 | 0.41 | 0.82 |
| | (2.89) | (2.35) | | (3.1) | (2.72) | | |
| Body and object use | 3.32 | 3.7 | 0.6 | 3.04 | 3.52 | 0.51 | 0.51 |
| | (2.75) | (2.74) | | (2.67) | (2.75) | | |
| Language | 2.44 | 3.1 | 0.23 | 2.14 | 2.93 | 0.13 | 0.93 |
| | (2.26) | (1.81) | | (1.94) | (1.84) | | |
| Social and self help | 3.28 | 3.45 | 0.75 | 3.21 | 3.41 | 0.75 | 0.71 |
| | (2.12) | (2.06) | | (2.08) | (2.44) | | |
| Total score | 16.22 | 16.82 | 0.8 | 15.11 | 16.36 | 0.64 | 0.95 |
| | (8.79) | (9.08) | | (9.14) | (10.16) | | |

SRS

| | | | | | | | |
|---|---|---|---|---|---|---|---|
| Social communication | 63.6 | 64.5 | 0.83 | 62.12 | 64.79 | 0.51 | 0.72 |
| | (15.54) | (16.5) | | (12.89) | (16.01) | | |
| Autism mannerisms | 30.53 | 31.1 | 0.75 | 30.04 | 30.33 | 0.88 | 0.82 |
| | (6.59) | (6.77) | | (7.52) | (6.47) | | |
| Social awareness | 23.59 | 20.73 | 0.02* | 23.46 | 20.62 | 0.04* | 0.74 |
| | (4.42) | (4.73) | | (5.29) | (4.67) | | |
| Social emotion | 19.41 | 19.53 | 0.92 | 19.25 | 19.25 | 1 | 0.98 |
| | (4.66) | (4.21) | | (4.66) | (4.88) | | |
| Total score | 138.23 | 137.25 | 0.89 | 132.72 | 137.96 | 0.46 | 0.43 |
| | (25.67) | (27.28) | | (23.37) | (25.98) | | |

CBCL

| | | | | | | | |
|---|---|---|---|---|---|---|---|
| Anxiety | 6.86 | 6.14 | 0.86 | 5.59 | 5.82 | 0.83 | 0.13 |
| | (5.2) | (4.43) | | (4.34) | (4.39) | | |
| Withdrawn | 4.45 | 4.59 | 0.35 | 4.26 | 4.43 | 0.71 | 0.81 |
| | (3.03) | (2.95) | | (2.7) | (3.02) | | |
| Somatic complaints | 2.44 | 3.08 | 0.98 | 2.63 | 2.36 | 0.97 | 0.65 |
| | (1.91) | (2.88) | | (2.63) | (2.51) | | |
| Internalization | 13.5 | 13.56 | 0.81 | 12.48 | 12.56 | 0.95 | 0.78 |
| | (8.63) | (8.13) | | (6.9) | (7.87) | | |
| Social problems | 7.83 | 7.59 | 0.32 | 7.7 | 7.63 | 0.2 | 0.44 |
| | (4.24) | (3.29) | | (4.07) | (3.81) | | |
| Thoughts problems | 5.96 | 7.37 | 0.59 | 5.08 | 6.81 | 0.95 | 0.41 |
| | (4.22) | (5.79) | | (4.37) | (5.39) | | |
| Attention problems | 10.54 | 11.07 | 0.77 | 11 | 10.93 | 0.98 | 0.25 |
| | (3.72) | (3.73) | | (4.2) | (4.18) | | |
| Rule-breaking behavior | 3.72 | 3.96 | 0.87 | 3.23 | 3.25 | 0.81 | 0.39 |
| | (3.38) | (2.79) | | (3.65) | (3.31) | | |
| Aggressive behavior | 8.46 | 8.76 | 0.73 | 8.22 | 7.82 | 0.89 | 0.87 |
| | (6.86) | (6.1) | | (6.44) | (6.09) | | |
| External | 12.08 | 12.96 | 0.89 | 11.42 | 11.07 | 0.45 | 0.83 |
| | (9.75) | (8.56) | | (9.6) | (8.67) | | |
| Total score | 51.04 | 52.14 | 0.86 | 45.17 | 50.5 | 0.83 | 0.29 |
| | (26.87) | (26.9) | | (23.02) | (24.42) | | |

SNAP-IV

| | | | | | | | |
|---|---|---|---|---|---|---|---|
| Inattention | 15.29 | 15.43 | 0.92 | 14.18 | 15.26 | 0.48 | 0.08 |
| | (5.58) | (5.14) | | (5.72) | (5.65) | | |
| Hyperactivity/impulsivity | 10.75 | 11.41 | 0.67 | 9.88 | 10.82 | 0.56 | 0.26 |
| | (5.75) | (5.96) | | (5.22) | (6.5) | | |
| Opposition/defiance | 9.26 | 7.38 | 0.21 | 7.71 | 7.32 | 0.77 | 0.03* |
| | (5.93) | (5.13) | | (4.75) | (5.44) | | |
| Total score | 34.85 | 34.5 | 0.93 | 31.88 | 33.59 | 0.68 | 0.02* |
| | (14.46) | (13.8) | | (13.83) | (15.99) | | |

The results are expressed as means (SD).
*P < 0.05.
ABC-T: Aberrant Behavior Checklist-Taiwan version;
CBCL: Child Behavior Checklist;
CGI-I: Clinical Global Impression-Improvement;
CGI-S: Clinical Global Impression-Severity;
SNAP-IV: Swanson, Nolan and Pelham (SNAP)-IV-Taiwan version;
SRS: Social Responsiveness Scale.

Further exploratory analysis within groups were implemented in different age groups of PS128 treatment and placebo (shown below in Table 4).

In the age group of from 7 to 15 years old (7˜15), four-week consumption of PS128 showed a trend of improved body and object use (P=0.04), nominal reduction of SRS total score (P=0.04), reduced anxiety (P=0.02) and rule-breaking behaviors (P=0.02), reduced hyperactivity and impulsivity (P=0.04), reduced opposition and defiance (ODD; P=0.045, shown as 0.05 in Table 4 as a result of rounded to 2 decimal places), and reduced SNAP-IV total scores (P=0.018, shown as 0.02 in Table 4 as a result of rounded to 2 decimal places).

In the age group of from 7 to 12 years old (7˜12), four-week consumption of PS128 showed that CBCL-anxiety (P=0.01), CBCL-rule-breaking behaviors (P=0.01), SNAP-IV-inattention (P=0.03), SNAP-IV-hyperactivity/impulsivity (P=0.02), SNAP-IV-opposition/defiance (P=0.02), and SNAP-IV total score (P=0.004) were further reduced. The reduction in behavioral abnormalities were even more obvious in the age group of from 7 to 12 years old (7˜12) with less p-values than those in the age group of from 7 to years old (7˜15).

TABLE 4
Exploratory analysis stratified by age groups for the
difference between baseline and week 4 of treatment

| PS128 | | | Placebo | | |
|---|---|---|---|---|---|
| Aged | Aged | Aged | Aged | Aged | Aged |
| 7~15 | 7~12 | 13~15 | 7~15 | 7~12 | 13~15 |

ABC-T

| | | | | | | |
|---|---|---|---|---|---|---|
| Sensory | 1.0 | 0.88 | 0.37 | 0.18 | 0.18 | N.C. |
| Relating | 1.0 | 0.72 | 0.21 | 0.84 | 1 | 0.39 |
| Body and object use | 0.04* | 0.06 | 0.48 | 0.5 | 0.5 | N.C. |
| Language | 0.33 | 0.48 | 0.37 | 0.31 | 0.39 | 0.39 |
| Social and self help | 0.4 | 0.46 | 0.62 | 0.78 | 0.78 | N.C. |
| Total score | 0.28 | 0.4 | 0.28 | 0.43 | 0.49 | 0.23 |

SRS

| | | | | | | |
|---|---|---|---|---|---|---|
| Social communication | 0.12 | 0.28 | 0.28 | 0.25 | 0.51 | 0.36 |
| Autism mannerisms | 0.08 | 0.21 | 0.13 | 0.19 | 0.19 | 0.89 |
| Social awareness | 0.93 | 0.9 | 0.7 | 0.41 | 0.62 | 0.52 |
| Social emotion | 0.09 | 0.33 | 0.08 | 0.29 | 0.38 | 0.55 |
| Total score | 0.04* | 0.13 | 0.15 | 0.2 | 0.36 | 0.4 |

CBCL

| | | | | | | |
|---|---|---|---|---|---|---|
| Anxiety | 0.02* | 0.01* | 0.82 | 0.38 | 0.45 | 0.6 |
| Withdrawn | 0.43 | 0.45 | 0.85 | 0.63 | 0.63 | 1 |
| Somatic complaints | 0.85 | 0.9 | 0.7 | 0.1 | 0.16 | 0.39 |
| Internalization | 0.12 | 0.12 | 0.8 | 0.15 | 0.2 | 0.39 |
| Social problems | 0.21 | 0.32 | 0.3 | 0.66 | 0.93 | 0.18 |
| Thoughts problems | 0.05 | 0.06 | 0.62 | 0.17 | 0.22 | 0.53 |
| Attention problems | 0.46 | 0.24 | 0.46 | 0.78 | 0.7 | 0.79 |
| Rule-breaking behavior | 0.02* | 0.01* | 1 | 0.11 | 0.14 | 0.39 |
| Aggressive behavior | 0.41 | 0.42 | 0.85 | 0.07 | 0.13 | 0.31 |
| External | 0.11 | 0.1 | 1 | 0.02* | 0.04* | 0.32 |
| Total score | 0.1 | 0.09 | 0.83 | 0.3 | 0.48 | 0.4 |

SNAP-IV

| | | | | | | |
|---|---|---|---|---|---|---|
| Inattention | 0.08 | 0.03* | 0.49 | 0.91 | 0.86 | 0.72 |
| Hyperactivity/impulsivity | 0.04* | 0.02* | 0.59 | 0.32 | 0.29 | 0.72 |
| Opposition/defiance | 0.05 | 0.02* | 0.78 | 0.77 | 0.79 | 0.33 |
| Total score | 0.02* | 0.004* | 0.61 | 0.86 | 0.96 | 0.46 |

Data expressed are P-values.
N.C.: difference equal to 0, the p-value is not calculable.
*P < 0.05.
ABC-T: Aberrant Behavior Checklist-Taiwan version;
CBCL: Child Behavior Checklist;
SNAP-IV: Swanson, Nolan and Pelham (SNAP)-IV-Taiwan version;
SRS: Social Responsive.

In fact, among the 31 subjects in the PS128 treatment group who have completed all the assessments at baseline and after 4 weeks of treatment, 14 individuals showed reduction of anxiety, as indicated by the decrease of CBCL score in anxiety, with the maximum score decrease from 12 to 4. In other syndromes evaluated by the CBCL scores, 15 individuals showed reduction of withdrawal issues, 10 individuals with less somatic complaints, 14 individuals with decreased score in social problems, 12 individuals with decreased score in thoughts problems, 11 individuals with less attention problems, 17 individuals with less rule-breaking behavior, and 11 individuals with less aggressive behaviors. In total, 14 individuals are shown to have reduced CBCL total scores, with a score decrease as large as 57 (decreased from 113 to 56).

The total ABC-T score that indicates behavioral problems in children with intellectual and developmental disabilities also decreases in 14 individuals out of 31 subjects. In the five subscales measured by ABC-T, 8 individuals showed reduction in problems related to sensation and perception; 9 individuals showed reduction in relating problems such as making relation and connection; 14 individuals showed reduction in abnormal body and object use behaviors such as rigid use of objects; 10 individuals showed improvement in communication and interaction; and 12 individuals showed improvement in adaptability and self-care.

For the assessment on social communication and interactions, restricted interests and repetitive behaviors, 14 individuals out of 31 subjects showed decrease in SRS total scores, with a largest decrease of 25 (from 139 to 114). For the four aspects of social communication and interactions assessed by SRS, 16 individuals showed improved social communication; 16 individuals show reduced autistic mannerisms; 12 individuals showed improved social awareness; and 15 individuals showed improved social emotion.

For the assessment of attention deficit, hyperactivity or impulsivity, and oppositional or defiance behaviors, SNAP-IV was used, and 18 individuals were found with decreased total score out of 31 subjects. Among them, 17 individuals showed improvement in attention deficit problems; 14 individuals showed less hyperactivity or impulsivity; and also 14 individuals showed less oppositional and defiance behaviors.

Actually, only one individual in the 31 subjects receiving the PS128 treatment for 4 weeks did not show any improvement or reduction of abnormal behaviors. That is, 30 individuals out of 31 subjects receiving the PS128 treatment for 4 weeks showed one or more improvement of the conditions or reduction of abnormal behaviors associated with neurodevelopmental disorders as assessed by the CBCL, ABC-T, SRS or SNAP-IV evaluation.

A total of 4 individuals (Subject ID: 1017, 7 years old; Subject ID: 1028, 7 years old; Subject ID: 1069, 8 years old; Subject ID: 1076, 7 years old) showed decreases in all the total scores of CBCL, ABC-T, SRS and SNAP-IV evaluations after receiving 4 weeks of the PS128 treatment. That is, all four evaluations indicated reduction of abnormal behaviors associated with neurodevelopmental disorders in these 4 individuals.

For example, Subject ID: 1064 (12 years old) showed a large decrease in the scores related to anxiety behavior evaluated by CBCL from 21 to 14, and showed less feelings of worthless or inferior, and also showed less worries, anxiety, fear, and guiltiness; the frequency of mentioning killing self was less, and the level of self-consciousness and embarrassment was less after taking the PS128 treatment for 4 weeks. Furthermore, the subject was also greatly improved in terms of less aggressive behaviors, with the CBCL score on aggressive behaviors decreasing from 26 to 8. The subject argued and teased less and did not threaten, get into fights and physically attacking others as often, destroyed less things either belonging to himself or others, demanded less attention, and screamed less. The subject was found to be in a more stable mood with less temper tantrums and less sudden changes in mood or feelings, and was less suspicious, stubborn or irritable.

Furthermore, Subject ID: 1064 was also noted for great improvement in social communication, showing less autism mannerisms such as stereotyped behaviors or interests. For examples, the subject showed less unusual ways of playing toys or other odd repeated behaviors such as hand flapping or rocking, less repeated talking or thinking of the same thing, less silly or strange behaviors, less unusual tones of voices or inappropriate laughs, better related to peers and play together appropriately, and cares more about not being “in step” with others. The subject was also found to show a bigger range of interests and not as sensitive to sounds, textures or smells, and was less teased or regarded odd or weird by others.

For another example, Subject ID: 1017 showed several improvements such as there is more the subject enjoyed in, complained less of loneliness, cries less, demands less attention, would feel guilty after misbehaving, not as jealous, less fears on certain animals, situations, or places other than school, less nervous or tense, less nervous movements or twitching, better liked by other kids, less constipated, less fearful or anxious, less overeating, less physical problems without known medical cause such headaches, less physical attacking of people, less preference being with older kids, not sleeping more than most kids during day and/or night, less stubborn, sullen, or irritable, less sudden changes in mood or feelings, less withdrawn and more involved with others.

In another evaluation, Subject ID: 1017 also showed more attention to social/environmental stimuli, hear better and less suspecting of a hearing loss, less strong reactions to changes in routine/environment, more responsive to other people's facial expressions and/or feelings, more reaction to painful stimuli such as bruises, cuts, and injections, less hurting of others by biting, hitting, kicking, etc., less repeating of phrases over and over, more imitation if other children at play, less twirling, spinning, and banging of objects, less repeating sounds or words over and over, less showing of “looks through” people, less destructive.

In a third evaluation, Subject ID: 1017 showed less “auto-pilot” when under stress, such as showing rigid or inflexible patterns of behavior that seem odd, was more able to recognize when others are trying to take advantage of him/her, less behaviors that seem strange or bizarre, more able to pick up on the meaning of conversations of older children or adults, less avoiding of eye contact, or less unusual eye contact, easier with changes in his/her routine, care more being “out of step” or not on the “same wavelength” with others, less regarded as odd or weird by other children, less upset in situations with lots of things going on, less difficulty in “relating” to adults, responded more appropriately to mood changes of others, less aimless wandering from one activity to another, less inappropriate or silly laughs, less difficulty in answering questions directly and ending up talking around the subject, less talking to people with an unusual tone of voice such as a robot or like he/she is giving a lecture, less emotionally distant and showing more of his/her emotions, less touching of others in an unusual way and less tense in social settings.

In a fourth evaluation, Subject ID: 1017 showed less frequency of losing things necessary for activities, less distracted by extraneous stimuli, less forgetful in daily activities, less fidgeting with hands or feet, less difficulty in playing or engaging in leisure activities quietly, less excessive talks, less impatient act such as blurting out answers before questions have been completed, less losing of temper, less arguing with adults, less blaming of others for his or her own mistakes or misbehavior, less touchy or annoyed by others, less angry and resentful, and less spiteful.

For another example, Subject ID: 1028 was more able to get his/her mind off certain thoughts, less confused, less crying, less daydreams or less getting lost in his/her thoughts, less harming of self, less destroying of things belonging to his/her family or others, able to eat better, getting along better with other kids, more able to feel guilty after misbehaving, less frequency of getting hurt, less accident-prone, more liked by other kids, less fearful or anxious, less overeating, more regular and normal sleeping pattern, less wetting the bed, less playing with own sex parts, less repeating of certain acts over and over, less refusal to talk, less screaming, less speech problems, less stubborn, sullen, or irritable, less sudden changes in mood or feelings, less temper tantrums, less thumb-sucking, more active, less unhappy, sad, or depressed, more active, less frequency of being unusually loud and less vandalism.

In another evaluation, Subject ID: 1028 showed improvements in hearing others, less hurting of others with biting, hitting and kicking, more responding to other people's facial expression and feelings, less severe temper tantrums or less frequency of minor tantrums, less repeating sounds or words over and over and less destructive.

In a third evaluation, Subject ID: 1028 was more able to pick up on conversations of older children or adults and understand the meaning, less clinging to adults, less awkward in turn-taking interactions with peers, better coordinated in physical activities, having facial expressions more matching to what he/she is saying, less regarded as odd or weird by other children, less showing of social awkwardness, better in making friends, being less regarded by other children as odd or weird, less social awkwardness, better “relating” to peers and adults, showing more understanding in cause and effect of events, less showing of overly serious facial expressions, less silly or inappropriate laughs, more able to answer questions directly and less ending up talking around the subject, more flexible and easier to change mind, less staring or gazing off.

In a fourth evaluation, Subject ID: 1028 was found to be better in organizing tasks and activities, better in engaging in tasks requiring sustained mental effort, more able to play or engage in leisure activities quietly, less showing of “always on the go” or “acting like driven by a motor”, less losing of temper, less touchy or being less annoyed by others, less angry and resentful and less spiteful or vindictive.

For another example, Subject ID: 1033 showed several improvements with less bowel movements outside toilet, less bragging or boasting, less clinging to adults or less dependent, less deliberate harming of self, less disobedient at home and at school, getting along with other kids better, less breaking of rules at home, school, or elsewhere, less fears of certain animals, situations, or places other than school, less showing of being nervous, high strung, or tense, less fearful or anxious, less physical problems without known medical cause such as rashes or other skin problems, less playing with own sex parts, better school work, less refusal of talking, less repeating of certain acts over and over, less running away from home, less screaming, less secretive, less strange behavior, less sulking, less thoughts about sex, less unusually loud talking, less wetting self during the day, less whining and less withdrawn.

In another evaluation, Subject ID: 1033 showed less strong reactions to changes in routine/environment, less acts of lunging and darting, better in following simple commands involving prepositions, less severe temper tantrums and/or less frequent minor tantrums, better in imitating other children at play, less showing of “looking through” people, less manipulating and less occupied with inanimate things, less involving in complicated “rituals” such as lining things up.

In a third evaluation, Subject ID: 1033 showed less behaviors in ways which seem strange or bizarre, more able to communicate his or her feelings to others, better coordinated in physical activities, more active in joining group activities, more willing to offer comfort to others when they are sad, less avoidance of starting social interactions with peers or adults, better personal hygiene, less wandering aimlessly from one activity to another, more able to understand how events are related to one another the way other children his/her age do, less overly serious facial expressions, more aware of times when he/she is talking too loud or making too much noise, less talking to people with an unusual tone of voice like a robot or as if he/she is giving a lecture, less reacting to react to people as if they are objects, giving less unusual or illogical reasons for doing things, less touching of others in an unusual way.

In a fourth evaluation, Subject ID: 1033 was able to better follow through on instructions and finish schoolwork, chores, or duties, less avoiding of engaging in tasks requiring sustained mental effort, less losing of things necessary for activities, less forgetful in daily activities, less leavings seat according to his/her own will, less excessive running about or climbing in situations that are inappropriate, less difficulty in playing or engaging in leisure activities quietly, less difficulty in awaiting turns, less interruption or intrusion on others, less blaming others for his or her mistakes or misbehaviors, less angry and resentful and less spiteful or vindictive.

For another example, Subject ID: 1069 showed improvements in many aspects including being able to concentrate more and pay attention for a longer time, being easier to get his/her mind off certain thoughts; less confused, better getting along with other kids, less fears of certain animals, situations, or places, other than school, less fears on he/she might think or do something bad, less feelings of he/she has to be perfect, less feelings of others are out to get him/her, less feeling of worthless or inferior, being teased less, less preference being alone than with others, less lying or cheating, less nervous, high strung, or tense, better liked by other kids, being less fearful or anxious, less too guilty feelings, less playing with own sex parts in public, less playing with own sex parts, less screaming, less self-conscious or embarrassed, less shy or timid, less blank stares, less storing of too many things he/she doesn't need, less strange ideas, less excessive talking, less teasing, less talking with unusual loudness, less whining, less withdrawn and more involved with others.

In another evaluation, Subject ID: 1069 was better at hearing others, more aware of surroundings, more visual reaction to a “new” person, less involvement in complicated “rituals” such as lining things up.

In a third evaluation, Subject ID: 1069 showed less in social situations than when alone, less “auto-pilot mode” such as rigid or inflexible patterns of behavior which seem odd when under stress, more interested in being with others, less behaves in ways which seem strange or bizarre, less clinging to adults, better self-confidence, less frustrating in trying to get ideas across in conversations, less unusual sensory interests, better personal hygiene, less socially awkward, less avoidance of people who want to be emotionally close to him/her, better in “relating” to adults, more imaginative and better at pretending without losing touch with reality, easier to separates from caregivers, better sense of humor and understood jokes better, better in knowing when he/she is too close to someone or is invading someone's space, less giving of unusual or illogical reasons for doing things, less touching of others in an unusual way, less tense in social settings, less stares or gazes off into space.

In a fourth evaluation, Subject ID: 1069 was better at sustaining attention in tasks or play activities, better in listening to others when spoken to directly, less avoidance of engaging in tasks requiring sustained mental effort, less distracting by extraneous stimuli, less forgetful in daily activities, less fidgeting with hands or feet or squirms in seat, less excessive running about or climbing that are inappropriate, better in playing or engaging in leisure activities quietly, less blurting out of answers before questions have been completed, better in awaiting turn, less interruption or intrusion on others such as butting into conversations/games, and less doing things deliberately that annoy other people.

For another example, Subject ID: 1076 showed improvements in many aspects including being more able to sit still, less restless, less hyperactive, eating better, less feelings of he/she has to be perfect, less feeling of others are out to get him/her, less feelings of worthless or inferior, being less teased, less hanging around with others who get in trouble, less nightmares, less fearful or anxious, better school work, not as preferring being with older kids, less stubborn, sullen, or irritable, less sudden changes in mood or feelings, less sulking, less talking or walking in sleep, less excessive talking, less temper tantrums or hot temper, being less unhappy, sad, or depressed, less whining and worries. In another evaluation, Subject ID: 1076 showed less strong reactions to changes in routine/environment, less severe temper tantrums and/or less frequent minor tantrums, better in waiting for needs to be met, more aware of surroundings and dangerous situations, less involvement in complicated “rituals” such as lining things up and less destructive. In a third evaluation, Subject ID: 1076 showed less behaviors that seem strange or bizarre, was more able to pick up on any of the meaning of conversations of older children or adults, was more able to communicate his or her feelings to others, was less awkward in turn-taking interactions with peers, was more able to imitate others' actions, was less regarded by other children as odd or weird, was able to get his/her mind off something easier once he/she starts thinking about it, was less avoiding people who want to be emotionally close to him/her, was able to focus more of his/her attention to where others are looking or listening, showed less overly serious facial expressions, was better in answering questions directly and not ending up talking around the subject, was less teased, was less inflexible, was easier to change his/her mind, had less unusual or illogical reasons for doing things, and was less tensed in social settings.

In another evaluation, Subject ID: 1076 was able to listen better when spoken to directly, less avoiding of engaging in tasks requiring sustained mental effort, less distracted by extraneous stimuli, less forgetful in daily activities, less fidgeting with hands or feet or squirms in seat, less excessive running about or climbing that are inappropriate, less showing of “always on the go” or of acts as if “driven by a motor”, less excessive talks, less blurting out of answers before questions have been completed, better in awaiting turn, less interruptions or intrusions on others less losing of temper, less arguing with adults, less active defying or refusing adult's requests or rules, less deliberate doing of things that annoy other people, less blaming of others for his or her own mistakes or misbehaviors, less touchy or annoyed by others, less angry, less resentful, and less spiteful or vindictive.

The foregoing descriptions of the detailed embodiments are only illustrated to disclose the principle and functions of the present disclosure and do not restrict the scope of the present disclosure. It should be understood to those skilled in the art that all modifications and variations according to the spirit and principle in the disclosure of the present disclosure should fall within the scope of the appended claims. It is intended that the specification and examples are considered as exemplary only, with a true scope of the disclosure being indicated by the following claims.

1-15. (canceled)

16. A method for reducing one or more behavioral abnormalities in a subject in need thereof, comprising administering a composition comprising an effective amount of Lactobacillus plantarum subsp. plantarum PS128 and a carrier thereof to the subject, wherein the subject is an individual aged between 7 and 15 years old and suffers from a neurodevelopmental disorder.

17. The method of claim 16, wherein the one or more behavioral abnormalities comprise withdrawal behavior, stereotyped behavior, repetitive behavior, compulsive behavior, aggressive behavior, rule-breaking behavior, deficit in social interaction, deficit in communication, deficit in attention, deficit in adaptability and self-care, deficit in social awareness, deficit in social emotion, abnormal sensation and perception, abnormal behavior in making relation and connection, abnormal body and object use behavior, hyperactive or impulsive behavior, oppositional or defiance behavior, or anxiety behavior.

18. The method of claim 16, wherein the neurodevelopmental disorder is anxiety, autism, autism spectrum disorder (ASD), a mental condition with a symptom of ASD, Tourette syndrome, obsessive compulsive disorder, attention deficit hyperactivity disorder, oppositional defiant disorder, Asperger's syndrome, childhood disintegrative disorder or Rett syndrome.

19. The method of claim 16, wherein the neurodevelopmental disorder is autism or autism spectrum disorder (ASD).

20. The method of claim 16, wherein reducing one or more behavioral abnormalities comprises reducing anxiety level, reducing abnormal body and object use behavior, reducing rule-breaking behavior, reducing hyperactive or impulsive behavior, reducing oppositional or defiance behavior, reducing attention deficit, or a combination thereof.

21. The method of claim 16, wherein the individual is aged between 7 and 12 years old.

22. The method of claim 21, wherein reducing one or more behavioral abnormalities comprises reducing oppositional or defiance behavior, reducing anxiety level, reducing rule-breaking behavior, reducing attention deficit, reducing hyperactive or impulsive behavior, or a combination thereof.

23. The method of claim 16, wherein reducing one or more behavioral abnormalities in the individual is determined by a standard behavioral evaluation.

24. The method of claim 23, wherein the standard behavioral evaluation comprises Autism Behavior Checklist-Taiwan version (ABC-T), Child Behavior Checklist (CBCL), Clinical Global Impression-Improvement (CGI-I), Clinical Global Impression-Severity (CGI-S), Swanson, Nolan and Pelham (SNAP)-IV-Taiwan version (SNAP-IV), Social Responsiveness Scale (SRS), or a combination thereof.

25. The method of claim 16, wherein the composition comprises Lactobacillus plantarum subsp. plantarum PS128 as a sole active ingredient for reducing the one or more behavioral abnormalities.

26. The method of claim 16, wherein the composition is orally administrated to the subject.

27. The method of claim 16, wherein the composition is a pharmaceutical compoition, and the carrier is a pharmaceutically acceptable carrier.

28. The method of claim 16, further comprising administering an antipsychotic in combination with the composition to the subject.

29. The method of claim 28, wherein the antipsychotic is selected from the group consisting of risperidone, aripiprazole, and atomoxetine hydrochloride.

30. The method of claim 28, wherein the antipsychotic and the composition are administered to the subject at different time intervals.

31. The method of claim 16, further comprising administering an additional therapy to the subject.

32. The method of claim 31, wherein the additional therapy is selected from the group consisting of applied behavior analysis (ABA), developmental, individual differences, relationship-based approach (DIR), treatment and education of autistic and related communication-handicapped children (TEACCH), picture exchange communication system (PECS), sensory integration therapy, Floortime approach and a combination thereof.

33. The method of claim 16, wherein the composition contains at least 1×10⁹ CFU of the Lactobacillus plantarum subsp. plantarum PS128.

34. The method of claim 16, wherein the composition is administered to the subject for at least 2 weeks.
